(19) United States
(12) Patent Application Publication
(10) Pub. No.: US 2007/0031972 A1
(43) Pub. Date: Feb. 8, 2007

(54) Detection of poisons in materials such as food using colorimetric detection

(76) Inventor: Amir J. Attar, Raleigh, NC (US)

Correspondence Address: INTELLECTUAL PROPERTY / TECHNOLOGY LAW, PO BOX 14329, RESEARCH TRIANGLE PARK, NC, 27709, US

(21) Appl. No.: 11/500,737

(22) Filed: Aug. 7, 2006

Related U.S. Application Data
(60) Provisional application No. 60/706,207, filed on Aug. 5, 2005.

(51) Int. Cl. G01N 33/20; G01N 33/50

(52) U.S. Cl. 436/80; 436/86; 436/90; 436/100

(57) ABSTRACT

The present invention relates to systems and methods for the rapid and reliable detection of acutely dangerous levels of poisons in liquid food and/or water samples. The systems preferably include an inexpensive and disposable laminated card including a dry chemical system of detection.

### CROSS-REFERENCE TO RELATED APPLICATION

The benefit of priority of U.S. Provisional Patent Application No. 60/706,207 filed Aug. 5, 2005 in the name of Amir J. Attar is hereby claimed under 35 USC 119.

### FIELD OF THE INVENTION

The present invention relates to detection cards or tabs that change color when contacted with poison-containing liquids, e.g., liquid foodstuffs or food extracts. Specifically, the present invention relates to a device, which can be part of a systematic procedure to detect dangerous amounts of poisons in foods, using said detection cards or tabs.

### BACKGROUND OF THE INVENTION

Although food poisoning is a well-established art, very few methods have been proposed to systematically determine if the food and/or water supply, have been poisoned prior to consumption. One reason for this lack of such detection systems relates to the fact that there exists a large number of poisonous species that can be used to artificially poison the food and water supply. Another complication associated with testing foodstuffs for poison is the need for rapid and reliable results.

Food poisoning, as a terrorist act, has become a real threat and its implementation a realistic possibility. Threats have been made to poison unsuspecting random people around the world. As such, we can no longer take for granted that the food and/or water we are consuming are free of artificial poison.

Although numerous poisons may be used to adulterate food, it is more probable that terrorists will use readily available, well-known poisons including, but not limited to cyanide, arsenic compounds, thallium compounds, sulfide and azide. This is especially true if the terrorists are planning a mass poisoning operation whereby a large amount of poison would be required.

Ironically, it is safe to assume that all foodstuffs contain traces of materials that are considered poisonous or even fatal if taken in large doses. Some of these materials may be present naturally as traces based on the nature of the foodstuff and/or where it originated from.

The objective of this invention is to determine if the food and/or water contains acutely dangerous amounts of poisons. In other words, the objective of the present invention is to detect poisons that were added to the foodstuffs intentionally and in quantities sufficient to poison people and other mammals in a relatively short time.

A useful method for detecting poisons in food should be quick, reliable, easily applied and the results unambiguously understood. In addition, it should be designed so that false negative and false positive errors are eliminated. From a practical point of view, the method and hardware should be relatively low in cost, stable and compact so that they can be widely disseminated to a wide range of users, both private and professional, and be readily available if it is suspected that the food and/or water supply has been poisoned.

Towards that end, the present invention relates to a methodology and systems to rapidly and reliably determine if the food and/or water supply contain acutely dangerous amounts of specific poisons.

### SUMMARY OF THE INVENTION

It is therefore an object of the present invention to provide an apparatus for the detection of acutely dangerous levels of poisons in food that is simple in construction, yet highly reliable.

A further object of the present invention is to provide an apparatus for the detection of acutely dangerous levels of poisons in food that is inexpensive and disposable.

In one aspect, the present invention relates to a colorimetric detection device for sensing the presence and identity of at least one poison in a liquid sample, said detection device comprising:
a support layer;
a color-forming chemical that changes color in response to exposure to said at least one poison, wherein the color-forming chemical is disposed on or in said support layer;
a cover encapsulating all outer surfaces of said support layer, except for at least one opening, wherein said at least one opening is a sufficient size to permit the introduction of droplets of liquid sample into the device to contact said color-forming chemical disposed on or in said support layer.

In another aspect, the present invention relates to a method of sensing the presence and identity of at least one poison in a liquid sample, said method comprising:
disposing a color-forming chemical on or in a support layer, wherein said color-forming chemical changes color in response to exposure to said at least one poison;
covering at least part of said support layer with a membrane;
encapsulating all outer surfaces of said support layer and membrane with a cover, except for at least one opening, wherein said at least one opening is a sufficient size to permit the liquid sample to enter the device and contact said color-forming chemical disposed on or in said support layer;
allowing the liquid sample to pass through said opening and said membrane so that the liquid sample will contact said color-forming chemical causing the same to change color; and
evaluating the resulting color of said color-forming chemical to determine the identity and/or concentration of said at least one poison,
wherein said liquid sample comprises a sample selected from the group consisting of water, liquid food an extract of solid food, extract of the content of the stomach, extract of feces, urine, ground water, waste water, wash water as well as industrial water.

Other aspects, features and embodiments of the invention will be more fully apparent from the ensuing disclosure and appended claims.

### BRIEF DESCRIPTION OF THE DRAWINGS

FIG. 1 is a cross-sectional view of the simplified embodiment of the poison detection card of the present invention.

FIG. 2 is a cross-sectional view of the simplified embodiment of the poison detection card of FIG. 1 after lamination.

FIG. 3 is a cross-sectional view of another embodiment of the poison detection card of the present invention.

FIG. 4 is a cross-sectional view of the embodiment of the poison detection card of FIG. 3 after lamination.

FIG. 5 is a cross-sectional view of yet another embodiment of the poison detection card of the present invention prior to lamination.

FIGS. 6A and 6B illustrate top and bottom views, respectively, of a simplified embodiment of the poison detection card of the present invention whereby the amount of the poison may be semi-quantitatively assessed.

FIGS. 7A and 7B illustrate top and bottom views, respectively, of the poison detection card of the present invention whereby the nature of the poison may be qualitatively assessed.

FIGS. 8A, 8B and 8C illustrate top and bottom views of an embodiment of the poison detection card of the present invention that can be analyzed in semi-quantitative tests using an electronic reader.

FIGS. 9A and 9B illustrate top and bottom views of an embodiment of the poison detection card of the present invention that can be used to detect poison in samples that need a high level of conditioning.

FIGS. 10A and 10B illustrate top and bottom views of an embodiment of the poison detection card of the present invention that includes two sample introduction ports having two different chromophores.

FIGS. 11A, 11B and 12 show a colorimetric detector according to one embodiment of the invention.

FIG. 13A, 13B, 13C and 13D show a colorimetric detector according to another embodiment of the invention.

FIG. 14A, 14B, and 14C show a colorimetric detector according to yet another embodiment of the invention.

FIG. 15A is a schematic representation of a colorimetric detector according to a further embodiment of the invention, prior to sample exposure.

FIG. 15B is a schematic representation of the colorimetric detector of FIG. 15A, after sample exposure.

FIG. 16A is a schematic representation of another colorimetric detector according to a further embodiment of the invention, prior to sample exposure.

FIG. 16B is a schematic representation of the colorimetric detector of FIG. 16A, after sample exposure.

FIG. 17A is a schematic representation of a colorimetric detector according to a still further embodiment of the invention, prior to sample exposure.

FIG. 17B is a schematic representation of the colorimetric detector of FIG. 17A, after sample exposure.

FIG. 18A is a schematic representation of a colorimetric detector according to yet another embodiment of the invention, prior to sample exposure.

FIG. 18B is a schematic representation of the colorimetric detector of FIG. 18A, after sample exposure.

FIGS. 19A, 19B, 20, 21 and 22 show detector cards according to further embodiments of the invention.

FIG. 23 shows a card with two strips and a QA pouch.

FIG. 24 shows a pouch before sealing, and FIG. 25 shows a pouch after sealing.

### DETAILED DESCRIPTION OF THE INVENTION. AND PREFERRED EMBODIMENTS THEREOF

The present invention relates to an apparatus and method of using dry chemical techniques to detect acutely dangerous levels of poisons in fluids such as food, food extracts and other fluids. The presence of poison is indicated when a color change from one color to another occurs. Other indicators, such as a change in fluorescence, may be used in special cases. As will be discussed herein, the present invention introduces several innovations to poison detection including, but not limited to, the use of dry chemical methodologies to rapidly and simultaneously detect a class of poisons, the elimination of interferences from food ingredients, simplification of result interpretation, qualitative and quantitative identification of the specific poison detected, and the introduction of a quality assurance (QA) process to verify that the detector is functioning properly, thus eliminating false negative and positive determinations.

The detection card of the present invention can be used in conjunction with the sample preparation and testing methodology described herein but is also an independent, stand-alone detection apparatus that may be used to detect other chemical species, poisonous or not. In addition, although the examples discussed herein utilize the methodology and apparatus to determine if foodstuffs have been poisoned prior to consumption, it is also contemplated that the methodology and apparatus may be used to test for poison contained in body fluids, e.g. for forensic purposes, environmental samples, industrial water, waster water, fluids from waste dumps, fluids from chemical processing facilities, etc.

It is noted that the objective of the present invention is to determine if a poison is present in the food at an acutely dangerous concentration and as such, the required sensitivity is much lower then may be required for corresponding analytical determinations of the same poisons in the environment. Further, the systems of the present invention reduce the need for time consuming sample-preparation procedures such as extraction or concentration and thereby provide for a more rapid test.

As defined herein, “food” and “foodstuffs” are used generically and include ingestible substances such as water, ice, ground water, all liquid and/or solid foods, substances used to cook liquid and/or solid foods (e.g., oils), substances used to flavor liquid and/or solid foods (e.g., spices and other powders), and the materials that the liquid and/or solid foods are cooked or washed in (e.g., cookware that may comprise leachable compounds). It is to be understood that reference to food and foodstuffs hereinafter is not meant to be limiting in any way.

In most embodiments, the present invention utilizes no electronic instruments, sensors computers, power sources and other ancillary resources and it does not require calibration. In other words, the presence of poison is detected colorimetrically using visual methods.

A quality assurance procedure (QA) has been built into the apparatus of all the embodiments as a method to reduce the number of false positive determinations and essentially eliminate the number of false negative determinations.

In another embodiment, the present invention requires the use of colorimetric instruments to determine the identity and the concentration of the poison in the food.

Although some of the chemistries described herein are known in the art, the methodology of their use, as well as the apparatuses in which they are incorporated, are new. The apparatus described herein eliminates or reduces interferences from various food components. Special verification tests were conducted to validate the applicability of the methodology and apparatus of the present invention to a wide range of various foods, cooking methods, cooking hardware etc. The apparatus is a flexible system that can be used to detect one poison or determine systematically if any poison, selected from a group of poisons, is present in the food. Furthermore, the results are unambiguously understood and as such, the methodology may be practiced by a wide range of users including lay people with only minimal training or knowledge of chemistry.

The methodology of the present invention, as described herein, is specifically directed to two groups of poisons, but may be easily expanded to include other groups of poisons as well. The two groups of poisons discussed herein are: anionic poisons, notably poisons that emit characteristic gases upon acidification including, but not limited to, organo-phosphonates, organo-arsenic compounds, carbamates, sulfides, cyanides, azides, sulphites, nitrites, heavy metals such as thallium, mercury, cadmium and lead salts, salts of actinides and lanthanides metals, arsenic compounds such as arsenites and arsenates, chromates, selenium compounds, as well as other organic poisons such as sulfur or arsenic mustards, lewisite etc.

The methods of the present invention have been designed to detect poisons in large or small quantities of food. The general guideline for detecting poison in food is to detect at least 50% of the LD₅₀ of the respective poison for a person who weighs 50 kg, with a safety margin of at least 20%. The apparatus described herein detects much smaller amounts of the poisons. In many cases the sensitivity of the system may need to be reduced to prevent unnecessary false positive determinations due to the natural presence of certain poisonous compounds in food.

The process of the present invention may include at least four technologies: extraction of solid foods (liquid foods may be used directly), screening tests for groups of poisons, validation of the presence of specific individual poisons, and a quality assurance process on the method of analysis.

Numerous approaches and methods are known for the extraction of specific components from food. For example, methods have been developed for extracting fats, carbohydrates or proteins from food using organic or aqueous solvents. When it comes to “conventional” poisons like cyanides, arsenates, thallium compounds etc., the type of food and the extraction process can greatly influence the extraction efficiency. Moreover, the extraction process dictates what other materials will also be extracted and thus, the types of interferences that may be experienced in the subsequent analytical process. Ultra fine extraction processes are well known in the art for detecting traces of heavy metals in foods, however, these processes will not result in the types of samples needed for a rapid determination of the presence of acutely dangerous amounts of poisons in food. Additionally, these ultra fine processes of the prior art require solvents and cannot be implemented quickly, even by a person skilled in the art of analytical detection. In light of the fact that the objective of the present invention is to detect acutely dangerous amounts of poisons, a relatively simple extraction process is preferred, bearing in mind that two variables must be controlled accurately during extraction, specifically the pH and the temperature.

By way of example, a simple extraction process using a surfactant and a buffer in water, which readily controls the types of components extracted and protects the other materials present in the sample, is disclosed in U.S. Pat. No. 5,783,399 issued Jul. 21, 1998 in the name of Mary Ann Childs et al.

It is noted that in order for a poison to be acutely dangerous to mammals, it has to be either water soluble or water miscible. Thus, extraction of the poison from a sample using an aqueous solution at a specific pH, optionally with a surfactant, should extract the majority of the poison. There are many water insoluble poisons in foodstuffs, however, their toxicity is expressed only after a minimum concentration is achieved over a much longer period of time.

The second technology described herein, i.e., screening tests, is used to detect or respond to multiple materials or poisons in a single test. Preferably, the apparatus of the present invention includes a chromophore, which changes to a unique color upon exposure to a unique poison. It is also contemplated herein that the present methodology and process may include a screening reagent that changes to the same or to different colors upon contact with different materials or poisons.

For example, it is well known in the art that many metal cations form a precipitate in the presence of sulfide ions, said precipitate corresponding to a variety of colors depending on the metal cation. This is often used to test for the presence of metal cations as well as to determine which metal cation may be present. Importantly, the selectivity and color may be affected by the pH or by other materials in the solution. These chemistries have been used in numerous qualitative analytical methodologies, however, heretofore have not been implemented in a dry chemical format for screening purposes.

The third technology described herein, i.e., validation of a positive result, is performed using dry chemical tabs including the screening reagent wherein the screening reagent responds specifically to the presence of a specific target poison by changing colors. Embodiments of these chemistries have been described previously by Fiegl et. al. (“Spot Tests in Inorganic Analysis,” Elsevier Pub. Comp., Amsterdam, (1972)), Feigl F. (“Spot Tests in Organic Analysis,” Elsevier Pub. Comp., Amsterdam, (1956)), Junreis, E. (“Spot Tests Analysis,” John Wiley and Sons, New York, (1985)), and Badcock, N. R. (“Detection of poisoning by Substances other than Drugs: A Neglected Art”, Am. Clin. Biochem., 37, 146-157, (2000)) using spot test plates or impregnated papers. Such tests utilize liquid reagents and often require that the reagents be freshly prepared right before use and/or require special pretreatment conditions or heating. In contrast, the validation tabs of the present invention eliminate the need for heating as well as the need to freshly prepare detection reagents.

In addition, the fourth technology described herein, i.e., quality assurance (QA), may be used to ensure that the detection card has operated properly, the chromophore is still effectively viable, and that no false negative or false positive occurred during the testing for poisons. The QA methodology has been designed to be fast and simple, so that the accurate testing of the foods for poisons can be completed in a relatively very short time. The QA process includes the addition of a known amount of poison-containing analyte to the apparatus to validate the sensitivity of the screening reagent and thus effectively verify the validation process.

One embodiment of the present invention corresponds to a colorimetric detection card, said detection card including a support layer having an amount of a chromophoric material therein or thereon. The chromophore may be dispersed on or in said support layer as micro- or nanoparticles on said support layer, embedded or impregnated in a thin polymeric film, or deposited on the surface of another solid material. The chromophoric material is selected so that it reacts with the target poison(s) to form a visible color change. Ideally, the color change is unique to the target poison or group of poisons.

The chromophores of the present invention may include a species selected from the group consisting of molybdates, phosphomolybdates, tungstates, phosphotungstates, iron salts such as sulfates, metallic sulfides such as zinc, calcium, barium, aluminum or strontium sulfides, organic materials such as 8-hydroxy-quinoline and its derivatives, 1-(2-pyidylazo)-2-napthol (PAN) and related compounds that include azo derivatives of heterocyclic compounds, rubeanic acid, diethyldithiocarbamate, dithizone, zincon, diphenylcarbazone, diphenylcarbazide (DPC) rhodizonic acid and its salts, titan yellow, cadion, functionalized diazonium salts including arsenic and phosphonic diazonium salts, triphenylmethane and xanthenes and other materials used in the spectrometric, fluorometric or colorimetric determination of species. Preferably, the chromophore includes a mixture of iron (II) and iron (III) sulfate compounds. The chromophoric mixture optionally includes acids, bases, preservatives, reactants, oxidizing agents, reducing agents, chelating agents, buffers, stabilizers, etc. The chromophore used herein for illustration purposes is a mixture of iron sulfates to detect cyanides, azides and sulfides.

The support layer of the detection card may be as simple as a sheet of paper or blotter paper or as sophisticated as microparticles of activated silica or alumina on a polymeric support wherein the chromophore is on or in the microparticulate surface. Other support layers include, but are not limited to, polymeric films, porous membranes, layered fibers, and metallic films The support layer may be chemically inert or it may be capable of assisting the reaction in some way. For example, the support layer may be acidic or basic. Other materials such as buffers, stabilizers or chelating agents may be incorporated within the chromophoric layer to facilitate the chromophoric reaction, prevent interferences, extend the shelve life of the chromophore, and increase its photostability. Importantly, the support layer must ensure maintenance of the chromophores on or in the support layer, must be physically and chemically capable of withstanding exposure to a variety of liquids, and must be non-reactive towards the chromophore and other ingredients in the chromophoric formulation. Optionally, the support layer may be liquid permeable.

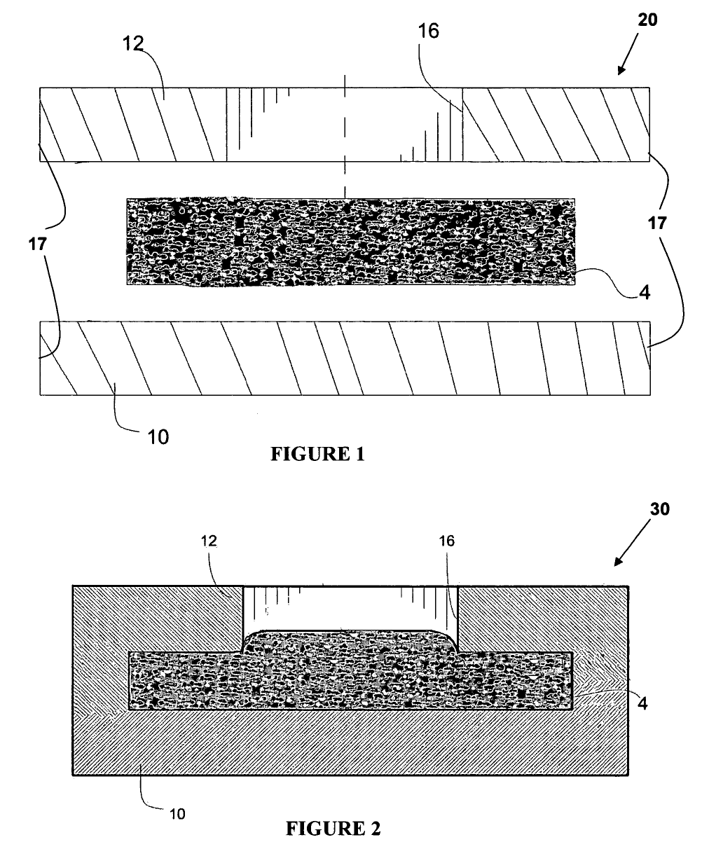

Referring to FIGS. 1 and 2, the cross-sectional view of an embodiment of the simplified detection card 20 is illustrated. The aforementioned support layer 4, including the chromophore thereon or therein, may optionally be coated with other materials such as soluble buffers or reactants that may remove specific interferants from the test solution. The support layer 4 is encapsulated between two transparent layers 10 and 12, wherein the two transparent layers 10 and 12 are preferably plastic or metallic laminatable plastics, as readily determined by one skilled in the art. One or more sample ports 16 are cut through one of the transparent layers with the number and placement of the holes dependent on the detection and QA methodology used to read the detection card 20. Written information identifying and/or quantifying the poison and any other useful information may be printed on the card, inserted between the laminated plastics, or printed on a label adhered to the card, when necessary. Upon lamination, the edges 17 of the transparent layers 10 and 12 are brought into contact with each other to seal the support layer 4 (see FIG. 2) to form a laminated detection card 30 having at least one sample port opening 16. Ideally, the support layer should be situated such that a user may view the color change from either side of the support layer.

In another embodiment of the present invention, the optional chemistries may be situated on a separate layer such as a hydrophilic membrane and assembled in parallel to the support layer having the chromophore thereon or therein. Importantly, the membrane must be physically capable of withstanding exposure to a variety of liquids and environmental gases. Moreover, the membrane must be non-reactive with the support layer and the chromophore. As defined herein, “membrane” denotes all permeable materials including, but not limited to, materials such as nylon, nitrocellulose, cellulose acetate, polysulfones, polycarbonates, polyesters, polyethylene, polypropylene and other poly-olefins, poly tetra fluoro ethylene, (PTFE), fluoropolymer membranes, thin sheets of fibers, etc.

This membrane may be included in the detection card for one or more reasons including, but not limited to, to filter out solid residue, to provide support for a masking material that reacts selectively with interferents and removes them, to provide support for materials that react and eliminate materials that can deactivate or consume the chromophore, to provide support for buffering materials that condition the sample before it reacts with the chromophore and to provide support for a species that may react with the poison whereby the product may be sensed more readily. Accordingly, different chemistries may be added on or in the permeable membrane to provide the desired effect to the detection process.

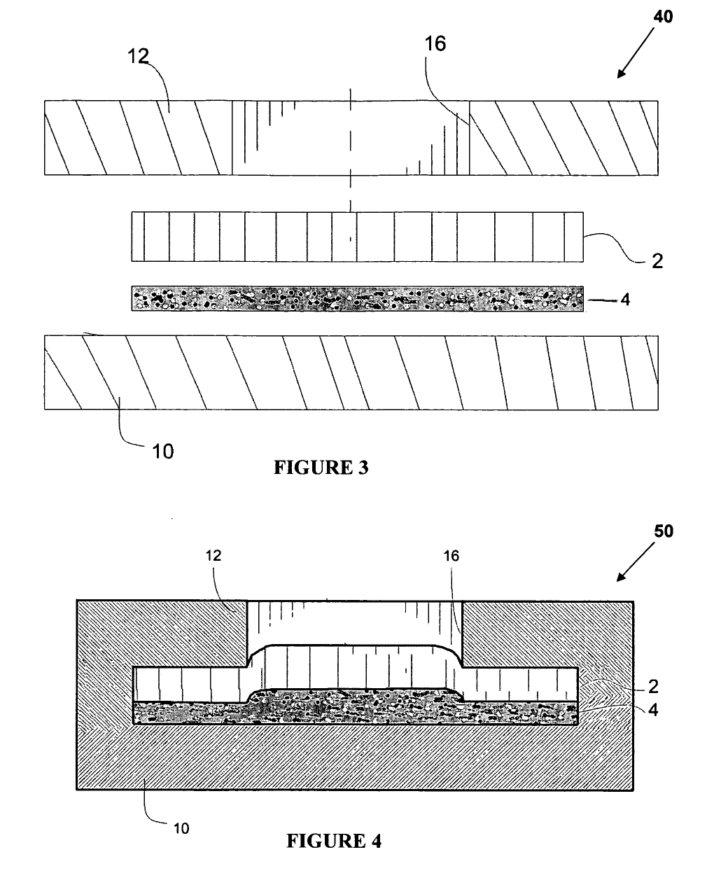

Referring to FIGS. 3 and 4, the cross-sectional view of this embodiment of the detection card 40 is illustrated. FIG. 3 includes a membrane filter 2 arranged in parallel with the support layer 4. Similar to FIGS. 1 and 2, the membrane 2 and the support layer 4 of FIG. 3 are encapsulated between two transparent layers 10 and 12, wherein the two transparent layers 10 and 12 are preferably plastic or metallic laminatable plastics. One or more sample ports 16 are cut through one of the transparent layers. Written information identifying and/or quantifying the poison and any other useful information may be printed on the card, inserted between the laminated plastics, or printed on a label adhered to the card, when necessary. When laminated, the transparent layers 10 and 12 are brought into contact with each other to seal the support layer 4 (see FIG. 4) to form a laminated detection card 50 having at least one sample port opening 16.

In yet another embodiment of the present invention, the chromophore is deposited on or in a support layer that is removed from the location of the sample introduction port. A permeable membrane may be positioned between the sample port and the support layer to filter out solids and other interfering materials, to host conditioning materials such as pH buffers, materials that remove selectively interfering materials, etc.

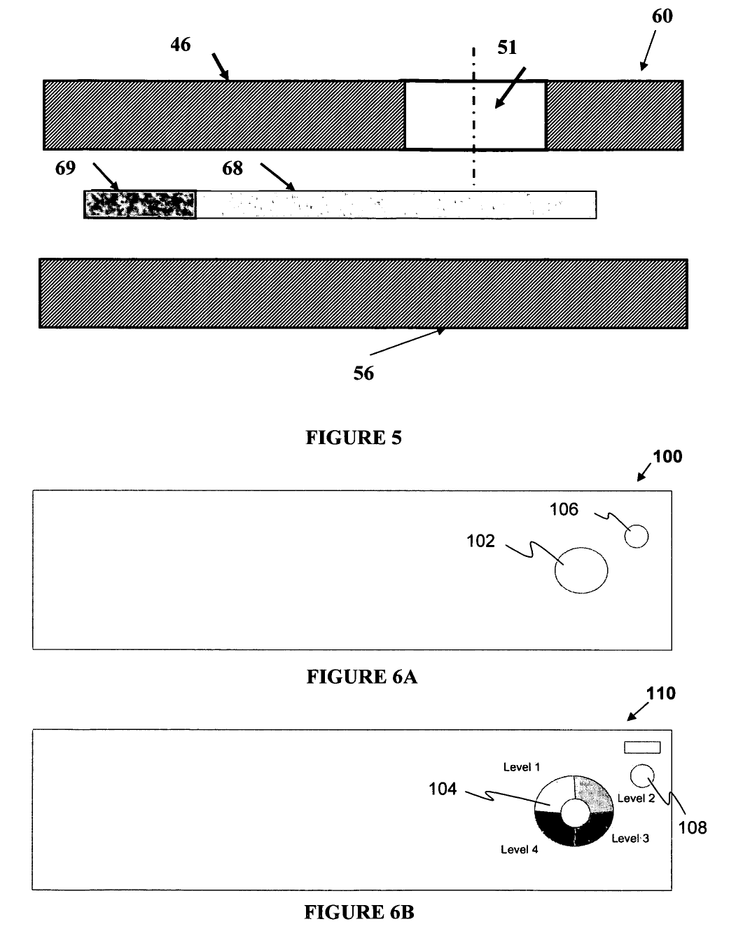

Referring to FIG. 5, the cross-sectional view of this embodiment of the detection card 60 is illustrated. A membrane filter 68 is arranged in series with the support layer 69. Similar to FIGS. 1-4, the membrane 68 and the support layer 69 of FIG. 5 are encapsulated between two transparent layers 46 and 56, wherein the two transparent layers 46 and 56 are preferably plastic or metallic laminatable or pressure adherent plastics. One or more sample ports 51 are cut through one of the transparent layers at a position other than the position of the support layer 69. Written information identifying and/or quantifying the poison and any other useful information may be printed on the card, inserted between the laminated plastics, or printed on a label adhered to the card, when necessary. Although not shown, the structure of FIG. 5 may be laminated. In practice, the fluid entering the sample port travels in a direction parallel to the planes of the transparent layers to the support layer having the chromophore thereon or therein and in the process undergoes a substantial amount of conditioning. This embodiment has the advantage of accommodating larger samples because of the longer path of filtering material.

The top and bottom views of the cards previously described and their relationship to the process described herein is introduced hereinbelow.

FIG. 6 illustrates the top and bottom views, respectively, of a simplified embodiment of the poison detection card of the present invention, specifically FIGS. 1-4. When a droplet of the sample is placed in the sample port 102, a characteristic color will form if one of the poisons to be detected is present in the sample. The color change may be viewed from the bottom side 110 of the detection card, and when no permeable membrane is present, or when the membrane is transparent, the color may be viewed from the top side 100 as well.

As previously introduced, the detection card may include written information instructing the user if and/or how much poison is present, when necessary. Referring to FIG. 6B, the poison concentration may be estimated in a semi-quantitative way by comparing intensity of the color change of the chromophore relative to a color chart 104 printed on the card. Importantly, if no color is detected at the sample port location, a test solution containing a known and detectable quantity of the poison(s) to be detected may be introduced into an optional QA port 106. If color is detected at the bottom side of QA port 106 (or the top side if no membrane is present), relative to the color chart 104, the user will know that the validation process is correct and the negative reading is a true negative (and not a false negative). It is noted that the internal structure of the QA port 106 is preferably analogous to that of the sample port, i.e., if no membrane is associated with the sample port, no membrane is associated with the QA port, etc. Importantly, the semi-quantitative assessment of the poison concentration can only be done provided the combination of the chemistry and structure of the poison(s) produces a unique, specific, and selective color. It also requires that the sample volume be fixed at the calibration value. Volumes that are too great or too small may cause the color produced to be non-uniform.

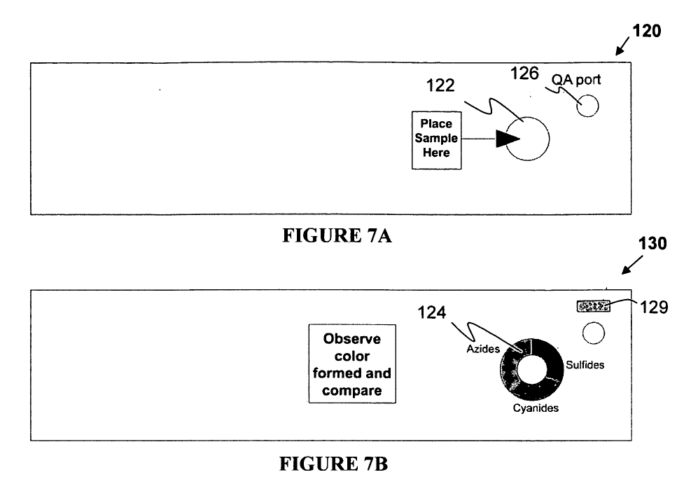

FIG. 7 illustrates the top and bottom views, respectively, of a simplified embodiment of the poison detection card of the present invention, specifically FIGS. 1-4. When a droplet of the sample is placed in the sample port 122, a characteristic color will form if one of the poisons to be detected is present in the sample. The color change may be viewed from the bottom side 130 of the detection card, and when no permeable membrane is present, the color may be viewed from the top side 120 as well.

As previously introduced, the detection card may include written information instructing the user if and/or how much poison is present, when necessary. Referring to FIG. 7B, the poison species may be identified by comparing the color change of the chromophore relative to a color chart 124 printed on the card. Importantly, if no color is detected at the sample port location, a test solution containing a detectable quantity of the poison(s) to be detected is introduced into the QA port 126. If color is detected at the bottom side QA port 126 (or the top side if no membrane is present), relative to the color chart 129, the user will know that the validation process is correct and the negative reading is a true negative (and not a false negative). It is noted that the internal structure of the QA port 126 is preferably analogous to that of the sample port, i.e., if no membrane is associated with the sample port, no membrane is associated with the QA port, etc. An example of this card includes a detection card for cyanides, azides and sulfides. The iron-based chromophore used in some of our examples forms blue, red and black colors with cyanides, azides and sulfides, respectively.

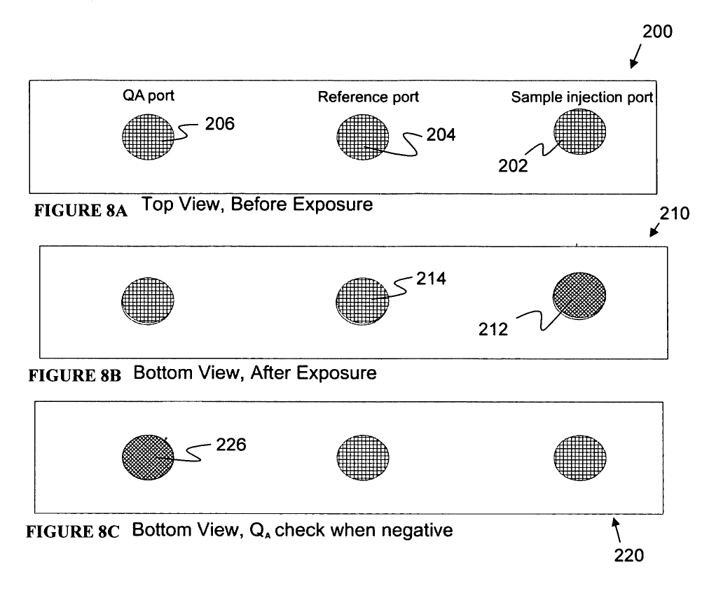

The detection card illustrated in FIG. 8 has three sample ports on the top side 200. The right port is the sample injection port 202, the middle port is a reference port 204 and the left port is a QA port 206 to ensure that a negative reading is not a false negative. When a sample is introduced to the sample injection port 202, color will appear on the bottom side of the sample port 212 if a poison is present in the sample. An electronic reader may be used to compare the color of the sample injection port 212 and the reference port 214 to quantitate the concentration of poison in the sample, as readily determined by one skilled in the art If no color is detected at the sample injection port 212, a drop of the QA solution may be introduced to the QA port 206 to ensure that the chromophore is still reactive. The appearance of color on the bottom side of the QA port 226 confirms that the chromophore is reactive and the negative measurement is a true negative.

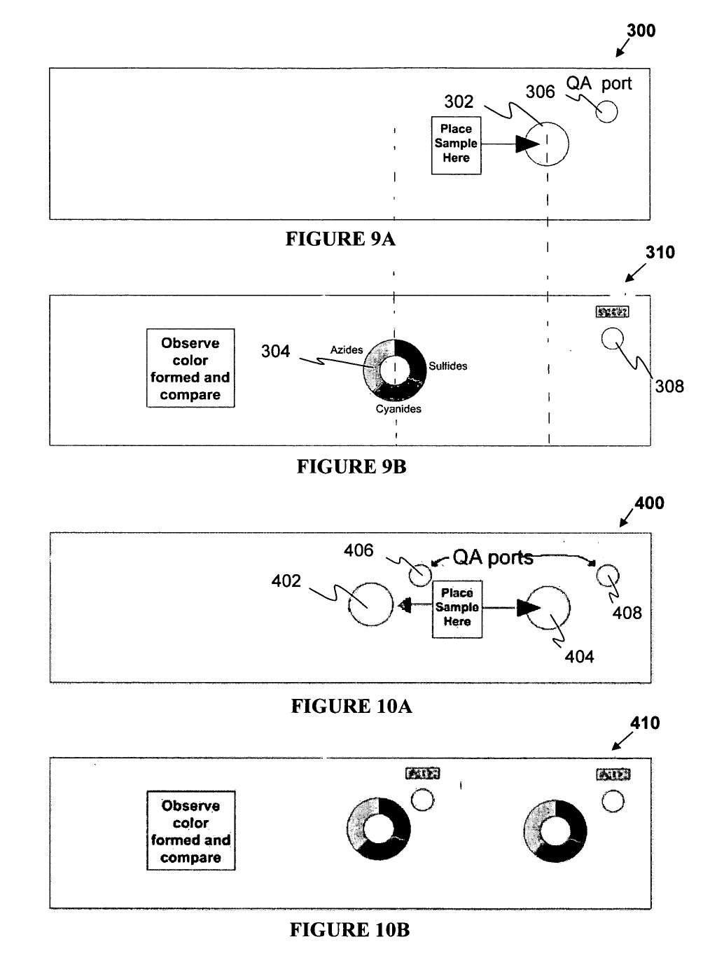

FIG. 9 illustrates the embodiment described herein whereby the support layer including the chromophore is located at some position other than the sample port position (see, e.g., FIG. 5). When a droplet of the sample is placed in the sample port 302, a characteristic color will form if one of the poisons to be detected is present in the sample. The color change may be viewed from the bottom side 310 of the detection card and when no permeable membrane is present, the color may be viewed from the top side 300 as well.

As previously introduced, the detection card may include written information instructing the user if and/or how much poison is present, when necessary. Referring to FIG. 9B, the poison species may be identified by comparing the color change of the chromophore relative to a color chart 304 printed on the card. Importantly, if no color is detected at the sample port location, a test solution containing a known and detectable quantity of the poison(s) to be detected is introduced into the QA port 306. If color is detected at the bottom side of the QA port 306, relative to the color chart 308, the user will know that the validation process is correct and the negative reading is a true negative (and not a false negative). It is noted that the internal structure of the QA port 306 preferably corresponds to that shown in FIGS. 1 and 2 or FIGS. 3 and 4. Although not illustrated, the detection card of FIG. 9 may include semi-quantitative information imprinted on the detection card.

FIG. 10 illustrates another embodiment of the present invention whereby the detection card includes two different sample ports 402, 404 including two different chromophores and two different QA ports 406, 408. For example, mercury and lead produce a black color in the presence of chromophoric sulfides but only mercury produces a violet color in the presence of the chromophore DPC. If a black color forms in the presence of sulfide, one can determine whether it is due to lead or mercury by introducing a second drop of the sample to a sample port including a support layer including DPC and looking for a violet color. More sample ports and chromophores may be added as needed to ensure that the identification of the poison is correct. For example, to determine whether a sample includes thallium, mercury, cadmium or lead, the detection card may include three sample ports each having a different chromophore. The resulting color changes, relative to a color chart, will allow the user to determine whether the sample includes Tl, Hg, Cd or Pb. Furthermore, the detection card may include written information informing the user how to determine if and/or how much poison is present, when necessary.

In yet another embodiment, the apparatus of the present invention may be sealed following manufacture for shipment. The detection card is preferably sealed in an envelope or laminated tab that are readily opened by the user with no tools. For example, the envelope or laminated tab may comprise metallic foil and/or polymeric film (e.g., polyethylene, polypropylene, polyester, etc.), said envelope or laminated tab including marks and/or labels instructing the user on how to open said envelope. In a preferred embodiment, the peelable tab covers at least one sample or QA port.

Another embodiment of the present invention is a kit comprising the detection card apparatus and instructions on how to use said apparatus to identify and/or quantify the poison in a liquid food sample. Optional components of said kit include, but are not limited to, a hand-held or small-sized instrumental colorimetric detector, a color chart for identification and/or quantification of the poison(s), at least one known sample for the quality assurance process, and extraction reagents and instructions relating to the extraction of poison(s) from food.

The features and advantages of the present invention are more fully shown by the following non-limiting examples.

### EXAMPLE 1

In this example, the following chromophore solution and supports A, B and C are used:
Chromophore solution: 0.5 gm NH₄Fe(SO₄)₂12H₂O in 10 ml DI Water.
Support A: Chromatography Paper #1 (e.g., Whatman CHR #1).
Support B: Chromatography Paper #3 (e.g., Whatman CHR #3).
Support C: Flexible Plates as used for TLC with 250 silica particles (e.g., Whatman PE SIL G).

The foregoing listing of specific products is intended to be illustrative only, and not to limit the usage or applicability of the invention. Other similar materials than specified can be used for the same functional purpose.

The chromophore is made by placing a small quantity of the solution on the support and air drying to remove the water. The typical quantity of solution used is 10 microliters, although any suitable amount of solution appropriate to the determination can be employed.

This chromophore is stable and responds calorimetrically to various poisons. Azides form a red color that gradually decays to yellow. Sulfides form a black color and cyanides form a blue color. The intensity of the latter depends on amount of ferric impurity in the solution and on the pH of the test sample. This chromophore can be provided on any of the supports A, B and C, or on any other suitable support or structural element.

### EXAMPLE 1A

### Color Viewed From Same Side as Sample Introduction Port

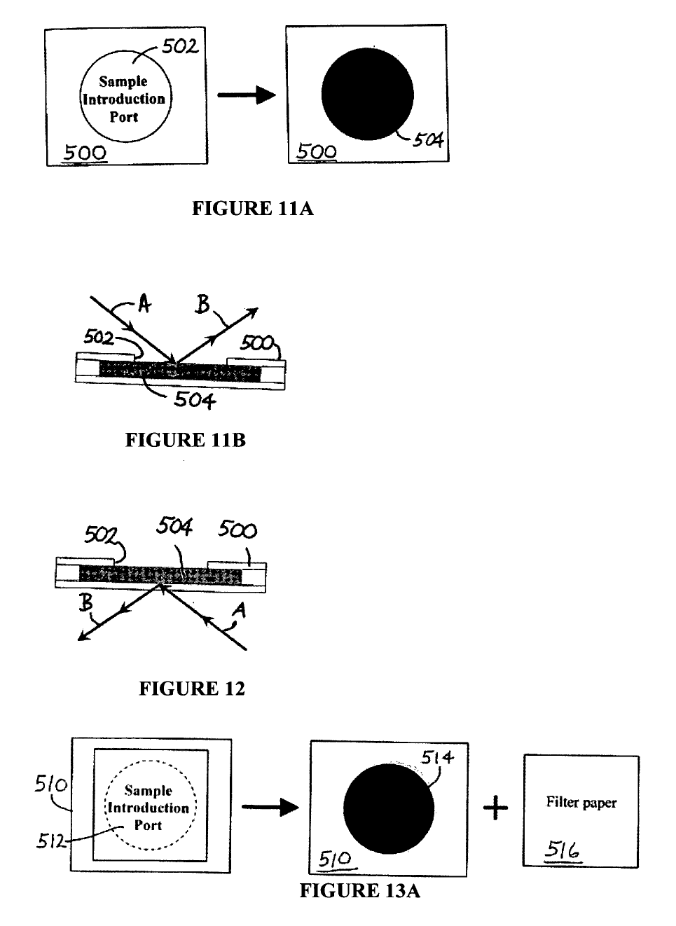

FIG. 11A in the left-hand portion thereof, shows a top section of the support 500 including a sample introduction port 502 therein. The right-hand portion of FIG. 11B shows the bottom section of the support, with the chromophore 504 mounted on the support for presentation of the chromophore to the sample introduction port 502. The top section of the support is mated with the bottom section, as indicated by the arrow between the left-hand and the right-hand portions of the drawing.

FIG. 11B is a cross-sectional elevation view of the chromophore assembly of FIG. 11A, showing incident radiation A and reflected radiation B from the chromophore 504, in which the colorimetric change is viewed from the top of the assembly.

### EXAMPLE 1B

### Color Viewed From Opposite Side to the Sample Introduction Port

FIG. 12 is a cross-sectional elevation view of a chromophore assembly of the type shown in FIG. 11B, but wherein the colorimetric change is viewed from the opposite side to the sample introduction port. The reference numbers in FIG. 12 correspond to those of FIG. 11B.

### EXAMPLE 1C

### Sample Introduction Through a Removable Filter on Top of Sample Port

If the sample contains a lot of food residue or other debris that can prevent viewing the color, one may use a temporary filter to remove the debris from obscuring the color. The filter materials may be paper, cotton, tea-bag material or any other suitable porous material. Some porous materials are also transparent and permit viewing the color formed from either side if the food debris does not interfere. The filter may contain:
I. a reagent, which reacts selectively with certain components of the sample and prevents them from reaching the chromophore; for example, nitrite may be employed on the filter to remove residues of azide and improve the detectability of cyanides.
II. a reagent, which conditions selectively one of the components to enable or enhance its chromophoric response.
III. a reagent, which conditions the sample to enable or enhance its chromophoric response; for example, an acidic buffer may be placed on the filter to improve the detectability of cyanides.

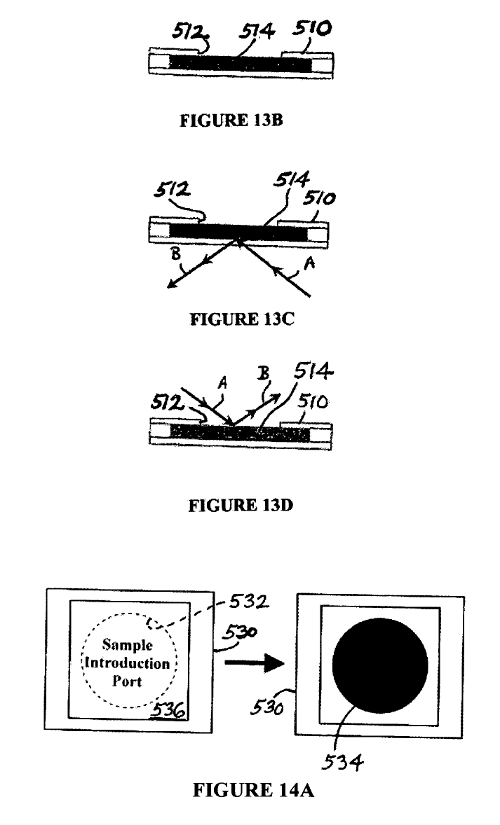

FIG. 13A in the left-hand panel shows a bottom section of the support 510 including a sample introduction port 512. The middle panel shows the bottom section of the support in the chromophoric detection assembly, with the chromophore 514 mounted on the support and presented to the sample introduction port for detection service. The right-hand panel of FIG. 13A shows a sheet of filter paper 516. The filter paper may be employed to remove debris that would interfere with the colorimetric detection.

FIG. 13B shows the colorimetric detection assembly from which the filter paper has been removed. The detection assembly includes the support 510, and the chromophore 514 being mounted for presentation and access through sample introduction port 512.

FIG. 13C shows a cross-sectional, elevation view of the assembly of FIG. 13B, in which the colorimetric changes viewed from the bottom of the assembly, with incident radiation A being converted to reflected radiation B.

FIG. 13D shows a cross-sectional, elevation view of the assembly of FIG. 13B, in which the colorimetric changes viewed from the top of the assembly, with incident radiation A being converted to reflected radiation B.

### EXAMPLE 1D

### Sample Introduction Through a Built-In Filter on Top of Sample Port

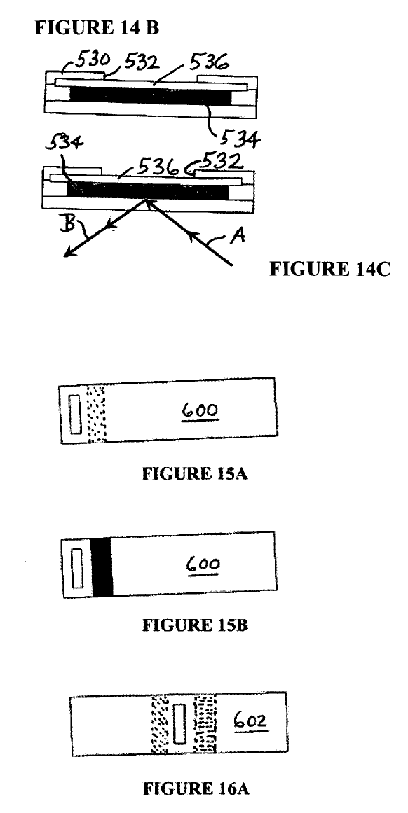

The colorimetric detection device in this example is shown in FIG. 14A, as comprising a support 530, the upper section thereof having a sample introduction port 532, and matably engageable with the upper section of the support. The lower section as shown in the right-hand panel of the drawing, has the chromophore 534 mounted thereon, for presentment to the sample through the sample introduction port 532.

This format thus uses a built-in laminated filter right under the sample introduction port, so that the chromophore is covered with the laminated filter. This filter serves the same function as the filter in the embodiment of FIG. 13A, but is not removed after the introduction of the sample. The filter material may be paper, cotton, tea-bag material or any other suitable porous material. This format is easier to use than the embodiment of FIG. 13A, but it allows viewing of the color formed mainly from the back side, although some filters may be sufficiently transparent to allow viewing through the top side as well. Any suitable additives may be used on the laminated filter, as hereinafter more fully described.

FIG. 14B shows a sectional elevation view of the assembled chromophore detector assembly, and FIG. 14C shows a cross-sectional, elevation view of the assembly, in which the colorimetric changes viewed from the bottom of the assembly, with incident radiation A being converted to reflected radiation B.

### EXAMPLE 2

### A Single Chromophoric Stain Placed Away From Sample Introduction Port

Strips of support similar to the ones described in Example 1 may be used as carrier of a chromophore. The difference between this example and Example 1 is that the chromophore is placed way from the sample introduction port and the colored stain is viewed from either side of the card. Filters may be placed on top of the sample introduction port as before and as before can optionally include materials that enhance the detection.

FIG. 15A shows a detector made with ammonium iron sulfate as before but placed 1.25 mm from the sample introduction port. FIG. 15B shows the stain after the chromophore has reacted with azide. One of the advantages of this format is that it allows eliminating the need for a filter in some cases, and the color formed may be viewed from either side of the card.

### EXAMPLE 3

### Two Single Chromophoric Stains Placed Away From The Sample Introduction Port in Two different Locations

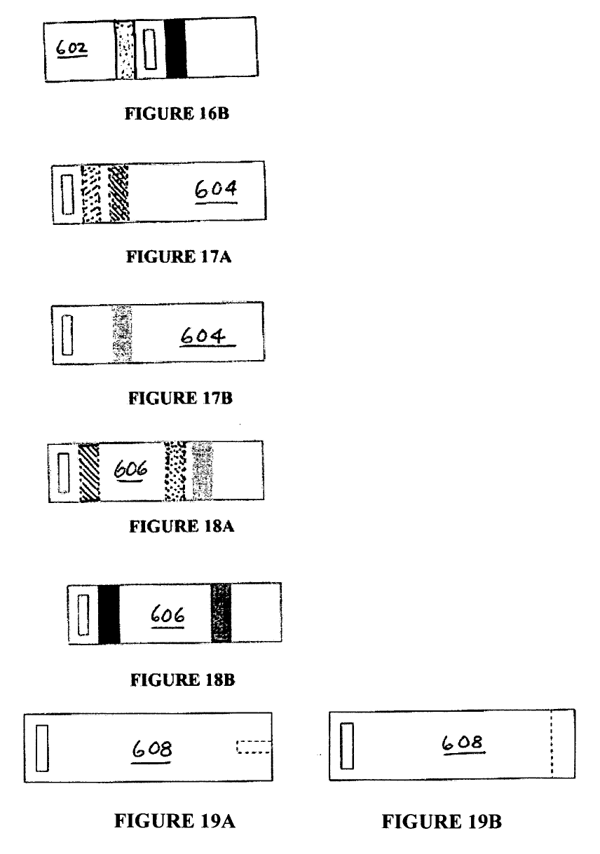

This format is similar to that described in Example 2 except that two or more reagents are placed on the support, in different directions away from the port. FIG. 16A shows two chromophoric stains placed 1 mm away from the sample port in opposite directions. One chromophore is a solution containing 0.22 grams of cadmium chloride in 50 ml water and the other is the same as used in Example 1. Ten microliters of each chromophore are used to stain the support and the water is dried. When a droplet of sample containing sulfides is introduced to the central port the liquid migrates in both directions and the sulfide forms a yellow color with the cadmium stain and a black color with the ferric salt. This combination of colors allows a more accurate detection as well as identification of the poison. FIG. 16B shows the card after it detected sulfide.

### EXAMPLE 4

### A Chromophoric Stain Placed Away From The Sample Introduction Port With a Preconditioning Reagent

This format is similar to that described in Example 2 except that two or more reagents are placed on the support. Cyanide is detected by putting on the support two reagents a copper salt and a benzidine derivative, such as O-Tolidine (OT). Ten microliters of solution containing 0.3 grams cupric sulfate, (CUS), in water are placed near the sample port, about 1 mm away, and 10 microliters of solution containing 0.3 grams OT in 10 ml 70% isopropyl alcohol (IPA) in water are placed on the support down from the copper stain and the sample port. The OT stain is not to touch the CUS stain. When a sample is placed in the sample port, it permeates through the support and encounters first the copper. The cyanide ions react with the copper and form a compound which continues to migrate with the liquid toward the OT stain. The complex reacts with the OT to form a blue color characteristic of the presence of cyanide. Oxidizing ions such as ferric, ceric and chromate ions interfere with the detection and will be recognized as cyanide. These ions can be removed using pre-treated filters. Many different cupric salts may be used instead of cupric sulfate, as well as different benzidine derivatives instead of the OT. These specific compounds are identified as being illustrated, and it is not intended thereby to restrict the applicability of the invention or disclosure herein. FIG. 17A shows a top view of such a detector before it reacts and FIG. 17B shows its appearance after it reacts.

### EXAMPLE 5

### End of Service Indicator-Using a Chromophore

Certain chromogenic reactions are slow and take one to three minutes to form color on the chromophore. In addition, the migrating front of the liquid is not always very visible. To simplify the detection of the analysis end, a test-end indicator, TEN, which changes its color when the analysis is completed, has been added. The TEN consists of two reagents placed in the path of the moving liquid front down flow from the chromophoric reagents. In the simplest embodiment, the first reagent is a simple acid or base and the second reagent is a pH indicator. Other arrangements have been used, such as a metallic stain followed by a chromogenic complexing agent, etc. The particulars of this description are not meant to limit the scope of the disclosure.

Ten microliters solution containing 0.3 grams citric acid in 10 ml water are placed 7 mm from the end of the detection strip downstream from the flow and the water dried.

Ten microliters of solution containing 0.01 grams methyl red in 10 ml 70% IPA are placed 4 mm from the end of the detection strip downstream from the flow and the water dried. When the fluid reaches the acid, it dissolves and carries some of it to the pH indicator. Once this solution reaches the pH indicator stain, it changes its color from yellow to red.

It will be appreciated that other acids and other pH indicators may be used in the same functions and that the quantities or concentrations used may be varied. In addition, the role of the acid may be fulfilled with a base. For example, 10 microliters of solution made by dissolving 0.3 grams of sodium carbonate in 10 ml water may be placed instead of the citric acid and 10 microliters solution containing 0.01 grams of phenol phthalein in 10 ml methanol. The migrating fluid will carry some of the basic carbonate with it and will turn the phenol phthalein red when it reaches it. Again, the naming of pH indicators, bases, solvents or quantities of materials involved is not meant to restrict the scope of the invention or disclosure.

FIG. 18 A shows a typical detector with an end of service indicator before it has reacted and FIG. 18 B shows the indicator after reaction has taken place.

### EXAMPLE 6

### Venting the Detector to Accelerate the Rate of Migration of the Test Solution

Adding a way for the air to exit the interior of the card causes the solution to migrate about twice as fast as when the detector is not vented. Several methods have been used to vent the card end.

One approach is to trim the end as shown in FIGS. 19A and FIG. 19B. Another approach is to incorporate within the laminate a short strand of porous material such as cotton or rayon to provide a way for the air to escape.

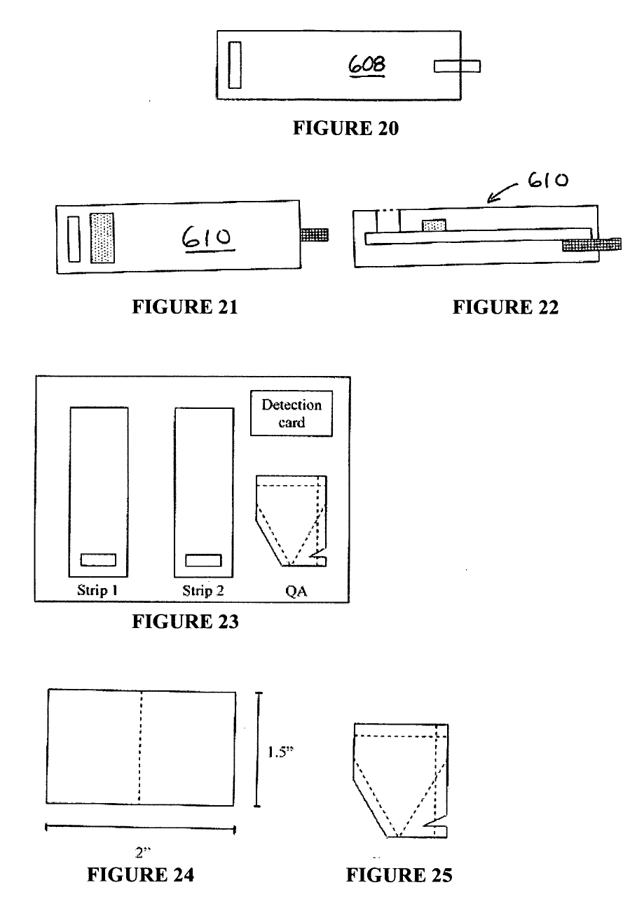

FIG. 20 shows an example of this structure. A ¾ inch strand of rayon, as used in knitting, was incorporated into the laminate and about 1-2 mm of it are allowed to extend out of the detection card.

The particular selection of method or material used to vent the card is not meant to restrict the scope of this invention. Many other methods and materials can be used for the same purpose.

### EXAMPLE 7

### Placing the Chromophore on a Separate Support Than the Liquid Carrier While All the Components are Incorporated Within a Laminate

A patch of support coated with a chromophore can be placed on top of the support to facilitate better flow when the coating blocks too much of the flow or when the chromogenic reaction product restricts the permeation or diffusion through the reacted chromophore. A patch of CHR #1 support, 7×14 mm, coated with ammonium ferric sulfate as described in Example 1 is placed on a strip of support 0-30 mm away from the sample introduction port. As the liquid migrates in the card and reaches the bottom of the patch, it diffuses in through the bottom of the patch and forms a visible color.

FIG. 21 is a top view of one such card and FIG. 22 shows a cross section of the card. Note that neither the dimensions nor the materials or the chromophore are intended to be construed as restricting the scope of this invention. Other materials may be used with different chromophores to accomplish the same goal. The chromophoric patch may be prepared as a separate component and incorporated within the detector at the time of assembly.

### EXAMPLE 8

### Quality Assurance Pouch and Its Inclusion With the Detectors

Many chromophores age and lose their chromogenic reactivity. Therefore, it is desired to provide a method to alert the user that the detector about to be used is functioning properly or not. This is done using a quality assurance (QA) pouch. To address this issue, a small pouch with a small quantity of known test reagent is attached to each detection card. Each detection card will thus include an additional detection strip to be used for QA purpose only. FIG. 23 shows a card with two strips and a QA pouch. One detector is to be used to test the sample and the second is to be used with a solution from the QA pouch. The results, mainly negative results, are to be rejected as possibly false negative results, if the QA detector does not respond.

Aluminum foil with polyethylene coating inside is used to make the 1″×1.5″ pouches. A small cotton ball is placed in each pouch and then 0.3 ml solution containing the reagent is added. The pouch is sealed thermally, trimmed diagonally and a small notch is made on the side of the pouch to facilitate tearing the pouch end before use. FIG. 24 shows a pouch before sealing and FIG. 25 shows it in a sealed state.

While the invention has been described herein in reference to specific aspects, features and illustrative embodiments of the invention, it will be appreciated that the utility of the invention is not thus limited, but rather extends to and encompasses numerous other variations, modifications and alternative embodiments, as will suggest themselves to those of ordinary skill in the field of the present invention, based on the disclosure herein. Correspondingly, the invention as hereinafter claimed is intended to be broadly construed and interpreted, as including all such variations, modifications and alternative embodiments, within its spirit and scope.

What is claimed is:

1. A colorimetric detection device for sensing the presence and identity of at least one poison in a liquid sample, said detection device comprising:
a support layer;
a first color-forming chemical that changes color in response to exposure to said at least one poison, wherein the first color-forming chemical is disposed on or in said support layer;
a cover encapsulating all outer surfaces of said support layer, except for at least one opening, wherein said at least one opening is a sufficient size to permit the liquid sample to enter the device and contact said first color-forming chemical disposed on or in said support layer.

2. The detection device of claim 1, characterized by at least one of the following:
(i) said support layer comprising a material selected from the group consisting of paper, modified paper, blotter paper, polymeric films, porous membranes, layered fibers, metallic films, and combinations thereof, and
(ii) the first color-forming chemical comprising at least one chromophore.

3. The detection device of claim 2, wherein the first color-forming chemical comprises at least one chromophore, wherein the chromophore comprises at least one compound selected from the group consisting of molybdates, phosphomolybdates, tungstates, phosphotungstates, iron sulfates, zinc sulfides, calcium sulfides, barium sulfides, aluminum sulfides, strontium sulfides, 8-hydroxy-quinoline and its derivatives, 1-(2-pyidylazo)-2-napthol (PAN), rubeanic acid, diethyidithiocarbamate, dithizone, zincon, diphenylcarbazone, diphenylcarbazide (DPC), rhodizonic acid and its salts, titan yellow, cadion, functionalized arsenic diazonium salts, functionalized phosphonic diazonium salts, triphenylmethane, xanthenes and combinations thereof.

4. The detection device of claim 1, wherein the first color-forming chemical comprises a mixture of iron sulfates.

5. The detection device of claim 1, wherein the support layer comprises at least one chemical species selected from the group consisting of acids, bases, preservatives, reactants, oxidizing agents, reducing agents, chelating agents, buffers, stabilizers, and combinations thereof.

6. The detection device of claim 1, wherein the at least one poison is selected from the group consisting of organophosphonates, organoarsenic compounds, carbamates, sulfides, cyanides, azides, sulphites, nitrites, thallium salts, mercury salts, cadmium salts, lead salts, actinide salts, lanthanide salts, arsenite salts, arsenate salts, chromate salts, selenium compounds, sulfur mustards, arsenic mustards, and lewisite.

7. The detection device of claim 1, wherein the first color-forming chemical is disposed on or in a microparticulate surface, wherein the microparticulate surface is disposed on the support layer.

8. The detection device of claim 1, wherein the cover comprises a plastic or metallic material, and optionally is transparent.

9. The detection device of claim 1, wherein the liquid sample comprises a sample selected from the group consisting of water, liquid food, extracts from solid food, ground water, industrial water, waste water, waste dumps fluids, and chemical processing fluids.

10. The detection device of claim 1, further comprising a membrane positioned between the first opening and the support layer.

11. The detection device of claim 10, wherein the function of the membrane is selected from the group consisting of: the filtration of solid residue; providing support for a masking material that reacts selectively with interferents; providing support for materials that react and eliminate species that can deactivate or consume the color-forming chemical; providing support for buffering materials that condition the liquid sample; providing support for a reactive species that may react with the at least one poison; and combinations thereof.

12. The detection device of claim 10, wherein the membrane comprises a hydrophilic species selected from the group consisting of nylon, nitrocellulose, cellulose acetate, polysulfones, polycarbonates, polyesters, polyethylenes, polypropylenes and other poly-olefins, poly tetra fluoro ethylene (PTFE), fluoropolymer membranes, thin sheets of fibers, thin sheets of glass fibers, and combinations thereof.

13. The detection device of claim 1, further comprising a transparent measurement area on said detection card located opposite said first opening in said cover for viewing the change in color of said first color-forming chemical as a result of contact by the at least one poison.

14. The detection device of claim 13, wherein the support layer is positioned between the transparent measurement area and the first opening.

15. The detection device of claim 1, further comprising a transparent measurement area on said detection card located at a position other than directly opposite said first opening in said cover for viewing the change in color of said first color-forming chemical as a result of contact by the at least one poison.

16. The detection device of claim 15, wherein the support layer is positioned in proximity to the transparent measurement area.

17. The detection device of claim 1, further comprising a quality assurance opening in said cover for allowing the known sample to enter the device and contact the first color-forming chemical disposed on or in a quality assurance support layer, optionally wherein the support layer and the quality assurance support layer are not in contact with one another, and optionally wherein the support layer and the quality assurance support layer are separated by a plastic or metallic material.

18. The detection device of claim 1, further comprising at least one additional opening in said cover for allowing the liquid sample to enter the device and contact a second color-forming chemical disposed on or in said support layer, wherein the second color-forming chemical may be the same as or different from the first color-forming chemical.

19. The detection device of claim 17, further comprising a comparative color chart on or below said cover adjacent to said transparent measurement area and including reference colors (i) indicative of predetermined levels of exposure to the at least one poison or (ii) indicative of the identity of said at least one poison.

20. The detection device of claim 1, characterized by at least one of (i) further comprising a sealable envelope for sealing the detection card for storage and shipment, and (ii) further comprising a peelable tab, said peelable tab sealing the at least one opening storage and/or shipment.

21. A method of sensing the presence and identity of at least one poison in a liquid sample, said method comprising:
disposing a color-forming chemical on or in a support layer, wherein said color-forming chemical changes color in response to exposure to said at least one poison;
covering at least part of said support layer with a membrane;
encapsulating all outer surfaces of said support layer and membrane with a cover, except for at least one opening, wherein said at least one opening is a sufficient size to permit the liquid sample to enter the device and contact said color-forming chemical disposed on or in said support layer;
allowing the liquid sample to pass through said opening and said membrane so that the liquid sample will contact said color-forming chemical causing the same to change color; and
evaluating the resulting color of said color-forming chemical to determine the identity and/or concentration of said at least one poison,
wherein said liquid sample comprises a sample selected from the group consisting of water, liquid food, extracts from solid food, stomach content extracts, feces extracts, urine, ground water, waste water, wash water, industrial water, and combinations thereof.

22. The method of claim 21, wherein the function of the membrane is selected from the group consisting of: the filtration of solid residue; providing support for a masking material that reacts selectively with interferents; providing support for materials that react and eliminate species that can deactivate or consume the color-forming chemical; providing support for buffering materials that condition the liquid sample; providing support for a reactive species that may react with the at least one poison; and combinations thereof.

23. The detection device of claim 1, characterized by at least one of:
(a) the color forming chemical being NH₄Fe(SO₄)₂12H₂O; and
(b) a filter being protectively arranged in relation to the color-forming chemical.

24. The detection device of claim 1, comprising a card, wherein the color change is viewable on at least one of (i) a same side of the card as said opening, and (ii) an opposite side of the card to said opening.

25. The detection device of claim 1, comprising a filter protectively arranged in relation to the color-forming chemical.

26. The detection device of claim 25, wherein the filter contains at least one of (i) a reagent that reactively removes components of the sample and prevents their reaching the color-forming chemical, (ii) a reagent to enhance chromophoric response of the color-forming chemical, and (iii) a reagent to condition the sample and increase its chromophoric response upon sensing by the color-forming chemical.

27. The detection device of claim 25, wherein the filter contains nitrite or an acidic buffer.

28. The detection device of claim 1, in combination in a kit including a quantity of known test reagent and quality assurance detection strip for use with the known test reagent.
